(19) United States
(12) Patent Application Publication
(10) Pub. No.: US 2026/0209062 A1
(43) Pub. Date: Jul. 23, 2026

(54) METHOD FOR PRODUCING METAL OXIDE NANOPARTICLES AND METAL OXIDE NANOPARTICLES PRODUCED THEREBY

(71) Applicant: University of Central Florida Research Foundation, Inc., Orlando, FL (US)

(72) Inventors: Sudipta Seal, Orlando, FL (US); Craig Neal, Orlando, FL (US); Elayaraja Kolanthai, Orlando, FL (US)

(73) Assignee: University of Central Florida Research Foundation, Inc., Orlando, FL (US)

(21) Appl. No.: 19/339,882

(22) Filed: Sep. 25, 2025

Related U.S. Application Data
(60) Provisional application No. 63/698,710, filed on Sep. 25, 2024.

(51) Int. Cl. C01F 17/32; C12N 5/00

(52) U.S. Cl. CPC C01F 17/32; C12N 5/525; C01P 2002/84; C01P 2004/04; C01P 2004/64; C01P 2006/22

(57) ABSTRACT

A method for producing metal oxide nanoparticles having high oxygen-defected surfaces and enhanced superoxide dismutase (SOD) activity, such as cerium oxide nanoparticles (CNPs) and hybrids thereof, which are useful for a variety of biomedical applications. The method is optimized such that the aging period of nanoparticle formation is reduced from the conventional aging period of weeks to a few days. This optimization enables industrial production of metal oxide nanoparticles with increased efficiency. Cerium oxide nanoparticles (CNPs) and hybrids thereof produced by the described method can be used for a variety of biomedical applications.

### FIELD OF THE INVENTION

The invention generally relates to the application of nanotechnology in design of nanomaterials for biomedical applications; particularly to a method for producing metal oxide nanoparticles having high oxygen-defected surfaces and enhanced superoxide dismutase (SOD) activity; and most particularly to optimization of this method to produce cerium oxide nanoparticles (CNPs) and hybrids thereof with increased efficiency and reduced production time.

### BACKGROUND

Nanoscale cerium oxide (nanoceria) has demonstrated substantial utility in biomedical applications and thus, there is an increasing interest in and demand for its fabrication. Among the various synthetic approaches, particles formed from an addition of hydrogen peroxide have shown unique functional and physicochemical character, suggesting an implication of the synthesis method in particle performance (e.g., free radical scavenging activity and modulation of oxygen metabolism).

The inventors have observed the influence of particle aging in an aqueous peroxide environment for up to 8 weeks and have identified three distinct material life stages (cluster/complex aggregation, condensation/hydrolysis, and mature particle redispersion) of the particles. Particles are seen to undergo initial partial hydrolysis, leading to sedimentation within 24 hours. After aging up to 4 weeks, the particles undergo a spontaneous redispersal into suspension with peroxy-phases transformed to oxide/oxyhydroxides, and the particles age to a stable final product within 6 to 8 weeks. Further, these life stages are correlated with pH changes, allowing the in-situ monitoring of particle aging and mapping of the particle physiochemical character (i.e., morphology, aggregation character, and colloid phase composition) onto a pH vs time relation. Colloidal phases formed during these stages are characterized and compared against the fully aged, bioactive particles.¹

These cerium oxide nanoparticles (CNPs) have shown substantial activity in several important biomedical applications (for example, in treatments of cancers and neurodegenerative diseases, and in diabetic wound healing), proving the strong industrial value of the CNPs.²⁻⁵ However, since formation of these particles occurs slowly over the course of approximately 6 to 8 weeks, practical application and scale-up of fabrication is difficult. Optimization of the method to produce cerium oxide nanoparticles (CNPs) with increased efficiency and reduced production time is urgently needed for widespread practical implementation of these nanoparticles in medicine.

### SUMMARY OF THE INVENTION

The invention provides this urgently needed optimized method for producing metal oxide nanoparticles having high oxygen-defected surfaces and enhanced superoxide dismutase (SOD) activity, such as, but not limited to, cerium oxide nanoparticles (CNPs) and hybrids thereof. The method is optimized to reduce the amount of time necessary for the aging period, during which nanoparticles are formed, from weeks to a few days. In the optimization, parameters of the method, such as temperature (heating), pH environment (titration), and atmosphere (oxygen concentration, aeration environment), are adjusted to accelerate hydrolysis and promote nanoparticle formation. Physicochemical analyses have confirmed the efficiency of the optimized method in producing a high yield of nanoparticles having high oxygen-defected surfaces and enhanced superoxide dismutase (SOD) activity that are suitable for various biomedical applications.

Aging of the particles with moderate heating/no heating promotes reactions and particle forming physiochemical processes without activating reconstructive processes that would modulate particle function in application. Overaging describes material reaction or processing times sufficiently long to allow these reconstructive processes to occur, even under the moderate conditions used. An overaging period that is too long may result in particles that are non-reactive, due to the reconstructive processes, but some overaging may contribute to beneficial stabilization of surface phases.

Shocking the particles refers to applying an intense driving force for particle formation. In the inventive method, synthesis would be a soft chemistry approach to metal oxide particle formation. Shocking the system can include, but is not limited to, increasing the pH to alkaline values or exposing to higher temperatures. Changing parameter values towards these extremes will hasten the synthesis process, but increasing too far will change the particle product material character. Specifically, this may result in particles which lose their colloidal stability (from being heated to a high temperature or titrated to a high pH, either of which would result in surface dehydration/hydroxylation and restructuring/surface deactivation).

In a most general aspect, the invention provides a method for producing metal oxide nanoparticles and hybrids thereof and metal oxide nanoparticles and hybrids thereof produced by the method.

In a general aspect, the invention provides accelerated production of metal oxide nanoparticles and metal oxide nanoparticles produced via the accelerated production.

In a general aspect, the invention provides a method for producing metal oxide nanoparticles in which particle formation is accelerated.

In a general aspect, the invention provides accelerated production of metal oxide nanoparticles having pharmaceutical benefits.

In yet another general aspect, the invention provides a method for producing cerium oxide nanoparticles (CNPs) in which particle formation is accelerated.

In a general aspect, the invention provides accelerated production of cerium oxide nanoparticles (CNPs) having pharmaceutical benefits.

In an aspect, the invention provides a method for producing metal oxide nanoparticles. The method includes forming a solution by adding an oxidizing agent to an aqueous metal oxide and heating the solution that was formed for a preselected time at a preselected temperature in a preselected atmosphere (aeration environment). The oxidizing agent is considered to be any substance capable of functioning as an oxidizing agent. Non-limiting examples of oxidizing agents include hydrogen peroxide, potassium dichromate, sodium hypochlorite, and calcium hypochlorite. The aqueous metal oxide can be, but is not limited to, a reducible rare earth metal oxide. Rare earth elements are the metals of the lanthanide series of the periodic table of elements. The lanthanides include, but are not limited to, lanthanum, cerium, praseodymium, neodymium, promethium, samarium, europium, gadolinium, terbium, dysprosium, holmium, erbium, thulium, ytterbium, and lutetium. The lanthanides, along with the elements of the radioactive actinide series, are referred to as inner transition metals. A preferred lanthanide series metal for nanoparticle production is cerium. Other non-limiting examples of aqueous metal oxides are actinide oxide and titanium oxide.

In another aspect, the invention provides a method for producing cerium oxide nanoparticles (CNPs). The method includes forming a solution by adding hydrogen peroxide to an aqueous cerium III salt and heating the solution formed for a preselected time at a preselected temperature in a preselected atmosphere (aeration environment). Cerium oxide nanoparticles are also known as nanoceria and/or as artificial enzymes capable of functioning as an antioxidant.

In an aspect of the method, heating the solution is carried out immediately after forming the solution or at a time period after forming the solution, such as, but not limited to about 24 hours after forming the solution.

In another aspect of the method, the aqueous metal oxide (non-limiting example, cerium) used for forming the solution is modified with a metal, such as, but not limited to, silver (Ag), to form a hybrid composition. The term “hybrid” refers to the fact that the particles contain more than one solid phase composition, for example, a metallic silver particle region and a cerium oxide-rich metal oxide region in a silver-modified cerium oxide (AgCNP) hybrid particle. Further, the term “hybrid” connotes differences in particle composition and physiochemical characteristics within a single formulation.

The term “modification” is used herein to indicate that chemical components beyond the metal oxide composition may not necessarily be doped into the metal oxide. With silver and cerium oxide, silver is only soluble within the cerium oxide lattice in very low amounts. However, silver can be formed at the cerium oxide surface to the extent that the silver-rich part of the particle can be similar or even greater in size relative to the ceria region of the particle.

A modifying metal can be, but is not limited to, a transition metal or a post-transition metal. The transition metals are found in the d-block of the periodic table and represent a “transition” of properties. Non-limiting examples of transition metals contemplated for the modification include copper (Cu), silver (Ag), iron (Fe), and zinc (Zn). A preferred, albeit non-limiting, transition metal for the modification is silver (Ag). The post-transition metals are found between transition metals and metalloids on the periodic table. A preferred, albeit non-limiting, post-transition metal for the modification is tin (Sn).

In another aspect of the method, the parameters or conditions of carrying out the method such as temperature (heating), pH environment (titration), and atmosphere (oxygen concentration, aeration environment), can be modified and/or manipulated to produce metal oxide nanoparticles having desired properties and/or characteristics. For example, when carrying out the method, the preselected time (for heating the solution) has a range between about 24 hours and about 96 hours and the preselected temperature (for heating the solution) has a range between about 40° C. and about 90° C. The term “preselected” refers to a selection that occurs prior to commencing the method. A non-limiting range of temperatures contemplated for use includes 40° C., 50° C., 60° C., 70° C., 75° C., 80° C., 85° C., and 90° C. A particularly preferred, albeit non-limiting range of temperatures includes from about 70° C. to about 90° C.

Non-limiting experimental examples of the preselected times and the preselected temperatures are a preselected time of about 24 hours and a preselected temperature of about 70° C.; a preselected time of about 96 hours and a preselected temperature of about 70° C.; a preselected time of about 24 hours and a preselected temperature of about 75° C.; a preselected time of about 96 hours and a preselected temperature of about 75° C.; a preselected time of about 24 hours and a preselected temperature of about 80° C.; a preselected time of about 96 hours and a preselected temperature of about 80° C.; a preselected time of about 24 hours and a preselected temperature of about 85° C.; a preselected time of about 96 hours and a preselected temperature of about 85° C.; a preselected time of about 24 hours and a preselected temperature of about 90° C.; and a preselected time of about 96 hours and a preselected temperature of about 90° C.

In carrying out the method, the solution formed is acidic or can be made acidic prior to heating such that the heating is carried out under acidic conditions. For example, the solution can be titrated to a specific acidic value prior to commencing heating of the solution. A non-limiting range of acidic value is a pH value of between about 2.0 and about 6.5. A preferred, albeit non-limiting, example of an acidic value is a pH value of about 2.5.

Since oxidation of cerium III to cerium IV is influenced by oxygen content in the atmosphere, manipulation of the air environment in which the method is carried out is advantageous for optimum particle formation. As used herein, aeration refers to the oxygen partial pressure: air composition. High oxygen partial pressures have been shown to influence average particle diameter and lattice parameters.⁶ The method can be carried out in ambient air but an artificial O₂/N₂ mixture-aeration environment is preferred, ranging from about 5% oxygen to 100% oxygen.

As used herein, the term “about” or “approximately” applies to all numeric values, whether or not explicitly indicated. These terms generally refer to a range of numbers that one of skill in the art would consider equivalent to the recited values (i.e., having the same function or result). In many instances these terms may include numbers that are rounded to the nearest significant figure.

As used herein, the terms “substantial” and “substantially” mean, when comparing various parts to one another, that the parts being compared are equal to or are so close in dimension that one skill in the art would consider them the same. Substantial and substantially, as used herein, are not limited to a single dimension and specifically include a range of values for those parts being compared. The range of values, both above and below (e.g., “+/−” or greater/lesser or larger/smaller), includes a variance that one skilled in the art would know to be a reasonable tolerance for the parts mentioned.

The terms “nanoparticles” and “particles” are used interchangeably herein; unless it is evident from the context that particles referred to are other than metal oxide nanoparticles produced by the described methods.

It should be understood that although the steps of the methods set forth herein can be carried out sequentially such steps are not necessarily required to be performed in the order described, and the order of the steps of such methods should be understood to be merely exemplary. Likewise, additional steps may be included in such methods, and certain steps may be omitted or combined in methods consistent with various embodiments of the present methods and/or nanoparticle compositions. Additionally, although the elements in the following method claims, if any, are recited in a particular sequence with corresponding labeling (unless the claim recitations otherwise imply a particular sequence for implementing some or all of those elements), those elements are not necessarily intended to be limited to being implemented in that particular sequence.

In another aspect, the invention provides metal oxide nanoparticles produced by the methods described herein, such as, but not limited to, cerium oxide nanoparticles (CNPs). These nanoparticles can have modifications, including but not limited to, modification with silver (Ag) to form hybrid compositions.

Since the described method enables accelerated particle formation, production efficiency is increased, thereby reducing production time. Thus, in another aspect, the methods described can be scaled up for industrial fabrication of the metal oxide nanoparticles in large quantities. Non-limiting examples of scaling up include preparing cerium oxide nanoparticles (CNPs) by scaling up the method for lab-scale synthesis to produce an approximately 50 ml (approximately 1.5 ounces) sample and/or by scaling up the method for bulk synthesis to produce an approximately 1-gallon sample. The metal oxide nanoparticles are contemplated for use in both research and biomedical/pharmaceutical applications. The metal oxide nanoparticles can be produced in amounts suitable for the intended application. Intended applications include, but are not limited to, biomedical applications such as treatment of diseases and wound healing. Non-limiting examples of such biomedical applications include treatment of cancer and neurodegenerative diseases, facilitating diabetic wound healing, and reducing effects of hypoxia, ischemia, transient ischemic attack (TIA), and stroke. The term “cancer” includes benign, malignant, and metastatic cancers and tumors. In a related aspect, the invention provides a composition comprising metal oxide nanoparticles and/or hybrids thereof produced by the methods described herein in an amount ranging from approximately 1.5 ounces to one gallon. A particularly preferred, albeit non-limiting, amount of the composition is at least one gallon or more.

In another aspect, the invention provides a method for treating a subject having a condition responsive to metal oxide nanoparticles including providing any of the metal oxide nanoparticles and/or hybrids thereof described herein and administering the metal oxide nanoparticles and/or hybrids thereof to the subject. The subject is preferably a human. The term “patient” can be used to refer to the subject and/or human. However, the inventive methods and metal oxide nanoparticles described herein are contemplated for administration to any animal or plant that can benefit therefrom. The condition responsive to metal oxide nanoparticles is at least one of, but not limited to, cancer (including benign tumors, malignant tumors, and metastatic tumors), a neurogenerative disease, a wound (particularly a wound associated with diabetes), and a condition causing hypoxia. Any condition causing hypoxia is contemplated for treatment; a particularly relevant condition is ischemia associated with a stroke or a transient ischemic attack (TIA).

In yet another aspect, the invention provides a method for conferring a cytoprotective effect on cells including providing any of the metal oxide nanoparticles and/or hybrids thereof described herein and administering the metal oxide nanoparticles and/or hybrids thereof to the cells. The cells are contemplated as including human, animal, and plant cells.

In another aspect, the components used to carry out the described methods, such as, but not limited to oxidizing agents, aqueous metal oxides, and metals can be packaged in containers and assembled in kits together with instructions for use.

The above-listed aspects are exemplary embodiments only and are not meant to limit the invention. Other objectives and advantages of this invention will become apparent from the following description taken in conjunction with the accompanying drawings, wherein are set forth, by way of illustration and example, certain embodiments of this invention. The drawings constitute a part of this specification and include exemplary embodiments of the present invention and illustrate various objects and features thereof.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

A more complete understanding of the present invention may be obtained by references to the accompanying drawings/data when considered in conjunction with the subsequent detailed description. The embodiments illustrated in the drawings are intended only to exemplify the invention and should not be construed as limiting the invention to the illustrated embodiments.

FIG. 1A is a photo of a sample of cerium oxide nanoparticles (CNPs) which were prepared by heating for 24 hours at 70° C.

FIG. 1B is a photo of a sample of cerium oxide nanoparticles (CNPs) which were prepared by heating for 96 hours at 70° C.

FIG. 1C is a photo of a sample of cerium oxide nanoparticles (CNPs) which were prepared by heating for less than 24 hours (ranging from 6 to 12 hours) at 90° C.

FIG. 2 is an ultraviolet-visible (UV-VIS) spectrophotometry graph generated with heat-treated cerium oxide nanoparticles (CNPs) and heat-treated silver-modified cerium oxide nanoparticles (Ag-CNPs).

FIG. 3A is a transmission electron microscopy image of a sample of cerium oxide nanoparticles (CNPs) that was heated for 24 hours at 70° C.

FIG. 3B is a transmission electron microscopy image of a sample of cerium oxide nanoparticles (CNPs) that was heated for 96 hours at 70° C.

FIG. 4 is a data table showing zeta potential measurements of heat-treated cerium oxide nanoparticles (CNPs) and heat-treated silver-modified cerium oxide nanoparticles (Ag-CNPs).

FIG. 5A is a graph showing superoxide dismutase (SOD) assay measurements of a sample of cerium oxide nanoparticles (CNPs) that was heated for 24 hours at 70° C.

FIG. 5B is a graph showing superoxide dismutase (SOD) assay measurements of a sample of cerium oxide nanoparticles (CNPs) that was heated for 96 hours at 70° C.

FIG. 5C is a graph showing superoxide dismutase (SOD) assay measurements of a sample of cerium oxide nanoparticles (CNPs) that was heated for 24 hours at 90° C.

FIG. 6 is a table recording the characteristic appearance of CNP samples after preparation and aging.

FIG. 7A is a transmission electron microscopy image of a sample of cerium oxide nanoparticles (CNPs) that was heated for 24 hours at 70° C.

FIG. 7B is a transmission electron microscopy image of a sample of cerium oxide nanoparticles (CNPs) that was heated for 96 hours at 70° C.

FIG. 7C is a transmission electron microscopy image of a sample of cerium oxide nanoparticles (CNPs) that was heated for 24 hours at 75° C.

FIG. 7D is a transmission electron microscopy image of a sample of cerium oxide nanoparticles (CNPs) that was heated for 96 hours at 75° C.

FIG. 7E is a transmission electron microscopy image of a sample of cerium oxide nanoparticles (CNPs) that was heated for 24 hours at 80° C.

FIG. 7F is a transmission electron microscopy image of a sample of cerium oxide nanoparticles (CNPs) that was heated for 96 hours at 80° C.

FIG. 7G is a transmission electron microscopy image of a sample of cerium oxide nanoparticles (CNPs) that was heated for 24 hours at 85° C.

FIG. 7H is a transmission electron microscopy image of a sample of cerium oxide nanoparticles (CNPs) that was heated for 96 hours at 85° C.

FIG. 7I is a transmission electron microscopy image of a sample of cerium oxide nanoparticles (CNPs) that was heated for 24 hours at 90° C.

FIG. 7J is a transmission electron microscopy image of a sample of cerium oxide nanoparticles (CNPs) that was heated for 96 hours at 90° C.

FIG. 7K is a transmission electron microscopy image of a sample of cerium oxide nanoparticles (CNPs) that was aged for 24 hours, pH adjusted to 2.5, and heated for 24 hours at 80° C.

FIG. 8A is a transmission electron microscopy image of a sample of silver-modified cerium oxide nanoparticles (Ag-CNPs) that was heated for 24 hours at 70° C.

FIG. 8B is a transmission electron microscopy image of a sample of silver-modified cerium oxide nanoparticles (Ag-CNPs) that was heated for 96 hours at 70° C.

FIG. 9A is a transmission electron microscopy image of a sample of cerium oxide nanoparticles (CNPs) that was heated for 24 hours at 80° C. This sample was prepared by scaling up the method for bulk synthesis to produce a 1-gallon sample.

FIG. 9B is a transmission electron microscopy image of a sample of cerium oxide nanoparticles (CNPs) that was heated for 24 hours at 80° C. This sample was prepared by scaling up the method for lab-scale synthesis to produce a 50 ml (~1.5 ounces) sample.

FIG. 10A is a graph showing superoxide dismutase (SOD) assay measurements of a sample of cerium oxide nanoparticles (CNPs) that was heated for 24 hours at 70° C.

FIG. 10B is a graph showing superoxide dismutase (SOD) assay measurements of a sample of cerium oxide nanoparticles (CNPs) that was heated for 96 hours at 70° C.

FIG. 10C is a graph showing superoxide dismutase (SOD) assay measurements of a sample of cerium oxide nanoparticles (CNPs) that was heated for 24 hours at 90° C.

FIG. 10D is a graph showing superoxide dismutase (SOD) assay measurements of a sample of silver-modified cerium oxide nanoparticles (Ag-CNPs) that was heated for 24 hours at 70° C.

FIG. 10E is a graph showing superoxide dismutase (SOD) assay measurements of a sample of silver-modified cerium oxide nanoparticles (Ag-CNPs) that was heated for 96 hours at 70° C.

FIG. 11 is a table showing the percentage of superoxide dismutase activity (SOD) activity calculated from SOD kinetic reaction graphs (time vs. absorption) of the samples shown in FIGS. 10A-E.

### DETAILED DESCRIPTION OF THE INVENTION

As required, embodiments are disclosed herein; however, it is to be understood that the disclosed embodiments are merely examples and that the methods and nanoparticles described below can be embodied in various forms. Therefore, specific structural and functional details disclosed herein are not to be interpreted as limiting, but merely as a representative basis for teaching one skilled in the art to variously employ the present subject matter in virtually any appropriately detailed structure and function. Further, the terms and phrases used herein are not intended to be limiting, but rather, to provide an understandable description of the concepts.

It can be advantageous to set forth definitions of certain words and phrases used throughout this disclosure. The terms “a” or “an”, as used herein, are employed to describe elements and components described herein. This is done merely for convenience and to give a general sense of the scope of the disclosure. This description should be read to include one or at least one and the singular also includes the plural unless it is obvious that it is meant otherwise. The term plurality, as used herein, is defined as two or more than two. The term another, as used herein, is defined as at least a second or more.

The term “communicate,” as well as derivatives thereof, encompasses both direct and indirect communication. The terms “include” and “comprise,” as well as derivatives thereof, mean inclusion without limitation. The term “or” is inclusive, meaning and/or. The phrase “associated with,” as well as derivatives thereof, can mean to include, be included within, interconnect with, contain, be contained within, connect to or with, couple to or with, be communicable with, cooperate with, interleave, juxtapose, be proximate to, be bound to or with, have, have a property of, have a relationship to or with, or the like. The phrase “at least one of,” when used with a list of items, means that different combinations of one or more of the listed items can be used, and only one item in the list can be needed. For example, “at least one of: A, B, and C” includes any of the following combinations: A, B, or C; A and B; A and C; B and C; and A, B, and C.

Note that not all activities described in the general description, or the examples are required, that a portion of a specific activity may not be required, and that one or more further activities can be performed in addition to those described. Still further, the order in which activities are listed is not necessarily the order in which they are performed.

For the purpose of promoting an understanding of the principles of the invention, reference will now be made to embodiments illustrated herein and specific language will be used to describe the same. It will nevertheless be understood that no limitation of the scope of the invention is thereby intended. Any alterations and further modification in the described methods and/or nanoparticles along with any further application of the principles of the invention as described herein, are contemplated as would normally occur to one skilled in the art to which the invention relates.

### Introduction

Cerium oxide nanoparticles (CNPs), synthesized by chemical methods to possess a substantial population of cerium sites in a reduced Ce⁺³ oxidation state, have been demonstrated to show substantial cytoprotective character in biological systems. Therefore, synthesis of such particles is highly desirable for use in biomedical applications. Similarly, properties such as high superoxide dismutase (SOD) activity, which affect cytoprotective character, may also be incorporated into commercial technologies. A particle formulation showing significant fractions of Ce⁺³, relative to oxidized Ce⁺⁴ states, and substantial SOD activity can be produced by adding an oxidizing agent (e.g., hydrogen peroxide) to an aqueous solution of Cerium (III) salt and aged for 8 to 10 weeks. During this aging period, cerium undergoes a hydrolysis process, sediments as a hydrous solid/gel, condenses, and redisperses as well-defined particles¹. A similar synthesis method has also been used for the synthesis of silver-modified cerium oxide nanoparticles (Ag-CNPs), which have been shown to have substantial anti-viral activities⁷. These Ag-CNPs undergo comparable age-specific processes to that of unmodified CNPs.

This invention describes a method for reducing the necessary aging period to no more than 2 to 5 days through additional processing steps for each of cerium oxide nanoparticles (CNPs) and silver-modified cerium oxide nanoparticles (AgCNPs) and can be extended to other nano-rare earth oxides with or without doping.

Doping is a standard technical term in general materials science. A dopant/doping agent is a solute element in a crystalline solid solvent (host lattice/material). Incorporation of dopants into a material can impart additional material functionality (for example, magnetic character) or change material properties/performance by modifying local crystal structure (for example, increasing lattice parameters and consequently lowering point defect formation energies). These changes alone or in conjunction with a modifying element (for example, an additional chemical element forming a surface segregated phase) can allow highly tuned performance by a complex material formulation.

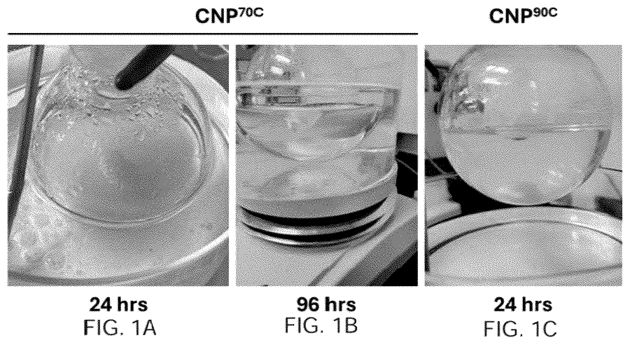

The additional processing steps of the inventive method include heating the solution immediately upon addition of hydrogen peroxide, heating following an initial aging period of 24 hours, or heating following titrating the aging solution to a pH value of approximately 2.5. Heating the solution is proposed to increase the rate of aging by providing thermal energy to drive hydrolysis and peroxide decomposition reactions mediated by the cerium species. Titration to pH near or about 2.5 is proposed to promote desorption of peroxide from formed particle surfaces, as well as to potentially mediate particle formation/dispersion from gel phase through the degradation of gel network-like structures. Images, as shown in FIGS. 1A-C, document the loss of color associated with terminal aging of cerium oxide nanoparticles as formed by peroxide addition at different time points and temperature treatments (e.g., FIG. 1A—CNPs heated to 70° C. for 24 hours: CNP^{70C}₂₄ₕᵣₛ). Associated physiochemical data suggest that particles produced by the proposed methods are similar in character to those produced via the conventional aging period. Thereby, the proposed methods successfully and substantially reduce required aging periods and drive nanoparticle synthesis through kinetically constrained processes at high throughput, without markedly altering particle physiochemical properties.

The occurrence of a kinetically constrained process, although energetically favorable, is infrequent. Heating the sample can promote the reaction/process rate and reduce the total required reaction time. A kinetically driven process has a physiochemical gradient/field applied to promote a kinetically constrained process, such as elevated temperature. Kinetics involve external influences that aid the reaction in forming nanoparticles, such as, but not limited to, stirring, sonication, aeration, and temperature.

### EXPERIMENTAL EXAMPLES

### Example A

### Synthesis of Cerium Oxide Nanoparticles (CNPs)

Cerium oxide nanoparticles (CNPs) were synthesized according to methods described in Neal et al.¹ and Heckert et al.⁸

Silver-modified cerium oxide formulations (AgCNPs) were synthesized according to methods described in Neal et al.¹

### Cerium Oxide Nanoparticles (CNPs) Heated Samples

Yellow-orange coloration of CNP particles heated for 24 hours at 70° C. (CNP^{70C}₂₄ₕᵣₛ) occurs from formation of peroxy-complexes with evolving cerium-rich phases (FIG. 1A). Upon aging, coloration reduces as peroxide is consumed or degraded by cerium species. Near complete loss of solution coloration occurs upon heating CNP solution for 96 hours at 70° C. (FIG. 1B) or for less than 24 hours at 90° C. (FIG. 1C).

Silver-modified cerium oxide formulations (AgCNPs) showed similar progression from a yellow-orange color to clear upon aging.

### UV-VIS Spectrophotometry

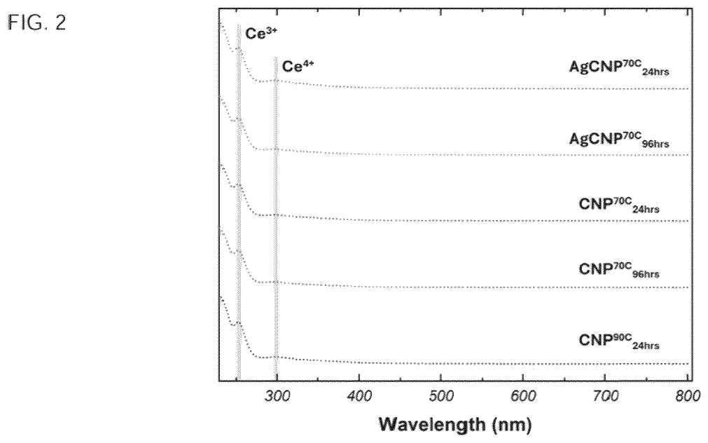

Ultraviolet-visible (UV-VIS) spectrophotometry of heat-treated cerium oxide nanoparticles (CNPs) and heat-treated silver-modified cerium oxide nanoparticles (Ag-CNPs) is shown in FIG. 2. Spectra for all measured samples are similar with no obvious peaks at wavelengths <600 nm and two peaks at cerium oxide related peaks, at ~252 nm and ~298 nm for Ce³⁺ and Ce⁴⁺, respectively.

### Transmission Electron Microscopy

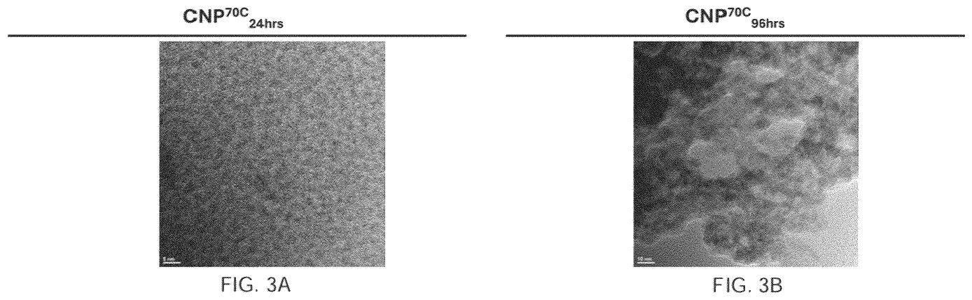

A sample of cerium oxide nanoparticles (CNPs) heated for 24 hours at 70° C. shows largely well-dispersed, approximately spherical particles ~2 to 3 nm in diameter (FIG. 3A). At 96 hours of heating time, some aggregates are observed with amorphous character and may be related to more extensive surface dehydration (FIG. 3B).

### Zeta Potential Measurements

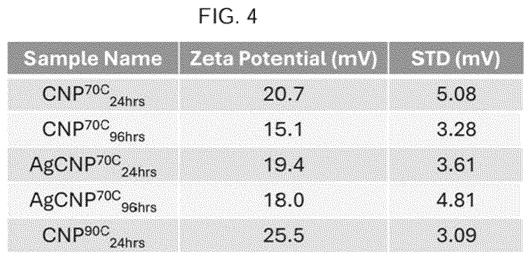

Particle zeta potentials are shown in the table of FIG. 4. Particle zeta potentials (reflective of particle surface charges and correlating with particle colloidal stabilities) are similar and near to a value of ~20 mV, suggesting moderate colloidal stability. Heating for 96 hours shows a slight decrease in zeta potential, relative to 24 hours, for both samples heated at 70° C., suggesting that longer periods of heating are contraindicated for optimal particle products. Heating to 90° C. for 24 hours resulted in particle products with the highest magnitude zeta potential values, suggesting the greatest extent of colloidal stability. Further, these particles were observed to age faster than any of the other samples. The faster aging is identified by loss of solution coloration from surface peroxy-complexation.

### Superoxide Dismutase (SOD) Assay

Measurements are performed to evidence radical scavenging capacity for particles following heat treatment. Great scavenging activity is demonstrated in the literature to correlate with cytoprotective effects upon biological cell treatment with nanoparticles.⁹ All tested samples possessed similar concentration-dependent activities suggesting that varying heat treatment procedures accelerate the aging process while keeping the surface active for high SOD activity.

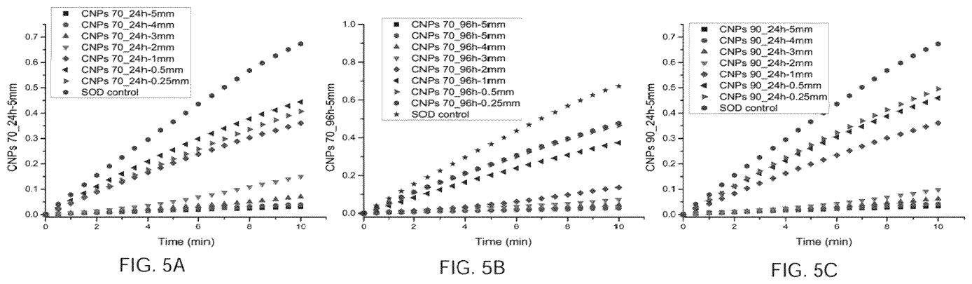

FIG. 5A is a graph showing superoxide dismutase (SOD) assay measurements of a sample of cerium oxide nanoparticles (CNPs) that was heated for 24 hours at 70° C. FIG. 5B is a graph showing superoxide dismutase (SOD) assay measurements of a sample of cerium oxide nanoparticles (CNPs) that was heated for 96 hours at 70° C. FIG. 5C is a graph showing superoxide dismutase (SOD) assay measurements of a sample of cerium oxide nanoparticles (CNPs) that was heated for 24 hours at 90° C.

### Example B

### Synthesis of Cerium Oxide Nanoparticles (CNPs)

Cerium oxide nanoparticles (CNPs) were synthesized according to methods described in Neal et al.¹ and Heckert et al.⁸

Silver-modified cerium oxide formulations (AgCNPs) were synthesized according to methods described in Neal et al.¹

### Cerium Oxide Nanoparticles (CNPs) Heated Samples

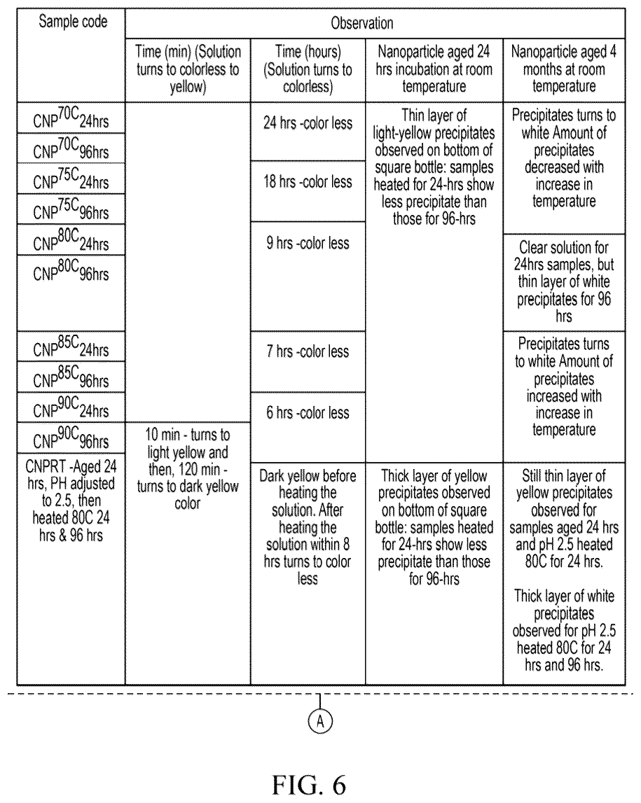

The characteristic appearance of the samples produced and aged are recorded in the table of FIG. 6.

### Transmission Electron Microscopy

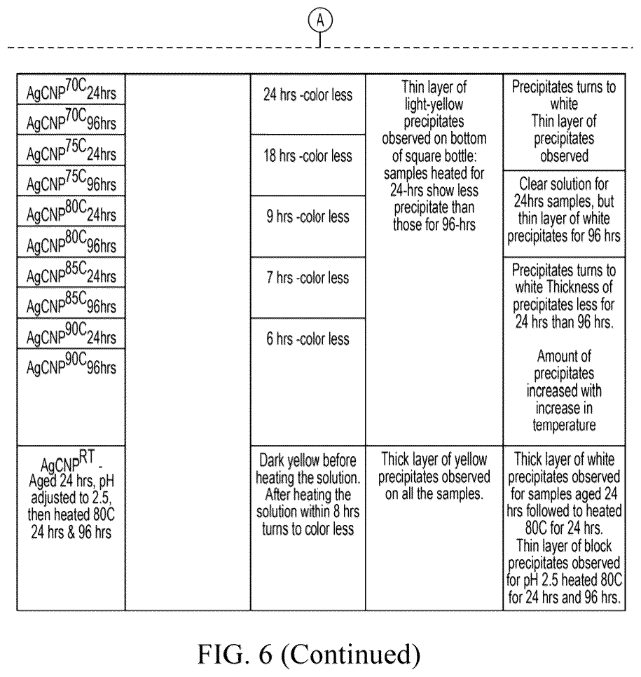

FIGS. 7A-K show transmission electron microscopy images of samples of cerium oxide nanoparticles (CNPs) produced by the methods described herein. The images shown are of heated-treated CNPs (particles) prior to undergoing the aging process, except for the sample shown in FIG. 7K, which was aged for 24 hours prior to heat treatment at 80° for 24 hours and 96 hours. Particles heated at 70° C. for 24 hours show largely well-dispersed, approximately spherical particles ~2 to 3 nm in diameter (FIGS. 7A-B). Some aggregates (heat-treated ~96 hours) are observed with more amorphous character and can be related to more extensive surface de-hydration. Similarly, particles heated at 75° C. (FIGS. 7C-D) and 80° C. (FIGS. 7E-F) for 24 hours and 96 hours exhibited some aggregation. In contrast, particles heated at 85° C. (FIGS. 7G-H) and 90° C. (FIGS. 7I-J) showed predominantly well-dispersed crystalline particles. Additionally, during synthesis, the nanoparticle solution pH was adjusted to 2.5 and maintained under continuous stirring at room temperature for 24 hrs. The particle size is 3 to 5 nm.

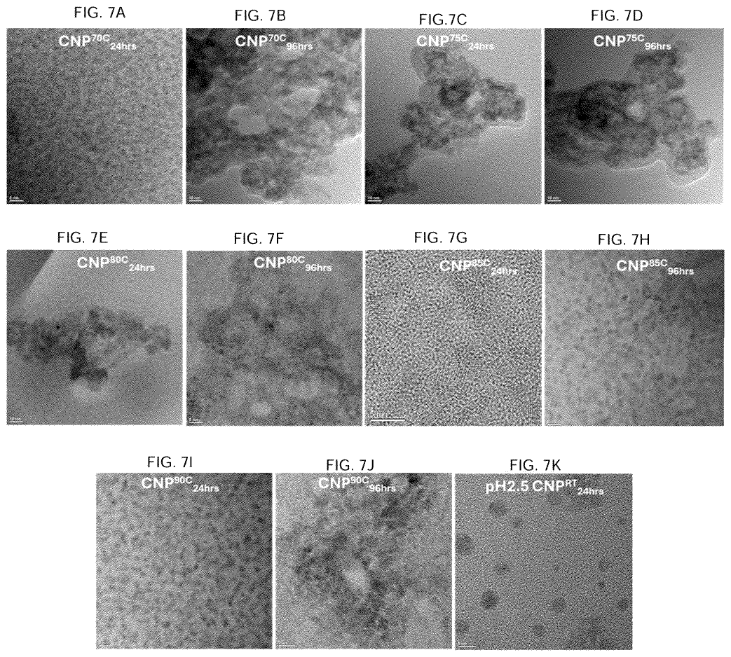

FIGS. 8A-B show transmission electron microscopy images of samples of silver-modified cerium oxide nanoparticles (Ag-CNPs) produced by the methods described herein. The images shown are of heated-treated Ag-CNPs (particles) prior to undergoing the aging process. Particles heated at 70° C. (FIG. 8A) for 24 hours show very well-dispersed, approximately spherical particles ~3 to 5 nm in diameter. In contrast, aggregates with crystalline particles are observed for particles heated at 70° C. (FIG. 8B) for 96 hours.

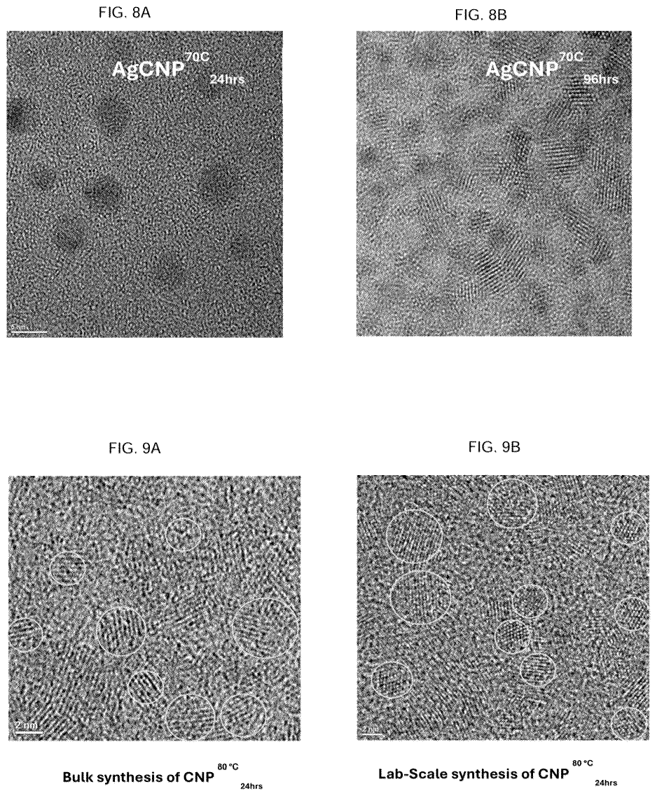

FIGS. 9A-B show transmission electron microscopy images of samples of cerium oxide nanoparticles (CNPs) produced by the scaled-up bulk synthesis methods described herein. The images shown are of heated-treated CNPs (particles) prior to undergoing the aging process. Particles heated at 80° C. for 24 hours show very well-dispersed, approximately spherical particles ~2 to 5 nm in diameter for both lab scale (50 ml (~1.5 ounces FIG. 9B) and bulk synthesis (1 gallon, FIG. 9A).

### Superoxide Dismutase (SOD) Assay

Measurements of heat-treated CNP samples were performed to evidence radical scavenging capacity for particles following heat treatment. Great scavenging activity is demonstrated in the literature to correlate with cytoprotective effects upon biological cell treatment with nanoparticles.⁹ All tested samples possessed similar concentration-dependent activities suggesting that varying heat treatment procedures accelerate the aging process while keeping the surface active for high SOD activity.

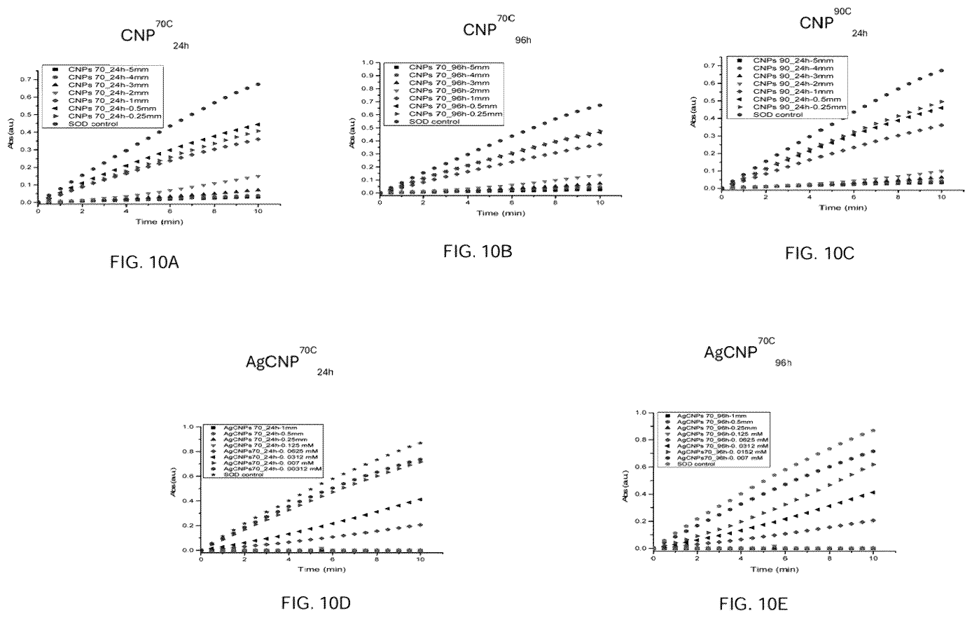

FIG. 10A is a graph showing superoxide dismutase (SOD) assay measurements of a sample of cerium oxide nanoparticles (CNPs) that was heated for 24 hours at 70° C. FIG. 10B is a graph showing superoxide dismutase (SOD) assay measurements of a sample of cerium oxide nanoparticles (CNPs) that was heated for 96 hours at 70° C. FIG. 10C is a graph showing superoxide dismutase (SOD) assay measurements of a sample of cerium oxide nanoparticles (CNPs) that was heated for 24 hours at 90° C. FIG. 10D is a graph showing superoxide dismutase (SOD) assay measurements of a sample of silver-modified cerium oxide nanoparticles (Ag-CNPs) that was heated for 24 hours at 70° C. FIG. 10E is a graph showing superoxide dismutase (SOD) assay measurements of a sample of silver-modified cerium oxide nanoparticles (Ag-CNPs) that was heated for 96 hours at 70° C.

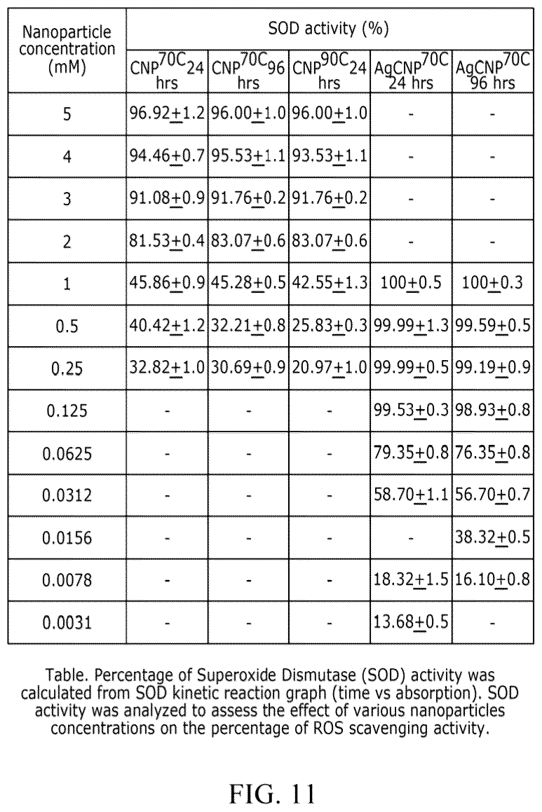

A table listing the percentage of superoxide dismutase activity (SOD) activity is shown in FIG. 11. These percentages were calculated from the SOD kinetic reaction graphs (time vs. absorption) of the samples shown in FIG. 10A-E. SOD activity was analyzed to assess the effect of various nanoparticles concentrations on the percentage of ROS scavenging activity.

### Conclusion

The long aging period for cerium oxide nanoparticles (from initial reaction to final product) of up to 10 weeks potentially prohibits their practical use in nanomedicine (non-limiting examples of such nanomedicine application are anti-cancer treatment, anti-neurogenerative disease treatment, and reduction of the effects of hypoxia, ischemia, stroke). The inventive methods described herein reduce particle aging time from up to 10 weeks to no more than 2 to 4 days. Thereby, synthesis of the nanoparticles is made scalable and more easily realized at commercial/industrial synthesis scales. The inherent value of the cerium oxide nanoparticles (CNPs) is their autocatalytic surface chemistry: producing free radical scavenging reactions which are known to be therapeutic towards a wide range of human pathologies. Further, the cerium oxide nanoparticle formulation, as produced by the methods described herein, has been shown to outperform most particles produced by similar methods, with respect to antioxidant/cytoprotective behaviors. This inventive synthesis method, without the processing/driving forces, outperforms similar methods. The additional processing steps enhance the rate and scale of production.^{10, 11} This method can be extended to other rare earth oxide materials in nanoscale, such as, but not limited to lanthanide oxides, Europium III oxide, Samarium III oxide, and Gadolinium III oxide.

All references cited herein are expressly incorporated by reference in their entirety. It will be appreciated by persons skilled in the art that the present disclosure is not limited to what has been particularly shown and described herein above. In addition, unless mention was made above to the contrary, it should be noted that all of the accompanying drawings are not to scale. There are many different features to the present disclosure and it is contemplated that these features may be used together or separately. Thus, the disclosure should not be limited to any particular combination of features or to a particular application of the disclosure. Further, it should be understood that variations and modifications within the spirit and scope of the disclosure might occur to those skilled in the art to which the disclosure pertains. Accordingly, all expedient modifications readily attainable by one versed in the art from the disclosure set forth herein that are within the scope and spirit of the present disclosure are to be included as further embodiments of the present disclosure.

The description in the present application should not be read as implying that any particular element, step, or function is an essential or critical element that must be included in the claim scope. The scope of patented subject matter is defined only by the allowed claims. Moreover, none of the claims invokes 35 U.S.C. § 112(f) with respect to any of the appended representative claims or claim elements unless the exact words “means for” or “step for” are explicitly used in the particular claim, followed by a participle phrase identifying a function.

Benefits, other advantages, and solutions to problems have been described above with regard to specific embodiments. However, the benefits, advantages, solutions to problems, and any feature(s) that can cause any benefit, advantage, or solution to occur or become more pronounced are not to be construed as a critical, required, sacrosanct or an essential feature of any or all the representative claims.

After reading the disclosure, skilled artisans will appreciate that certain features are, for clarity, described herein in the context of separate embodiments can also be provided in combination in a single embodiment. Conversely, various features that are, for brevity, described in the context of a single embodiment, can also be provided separately or in any sub-combination. Further, references to values stated in ranges include each and every value within that range.

The above discussion is meant to be illustrative of the principles and various embodiments of the present invention. Numerous variations and modifications will become apparent to those skilled in the art once the above disclosure is fully appreciated. It is intended that the following representative claims be interpreted to embrace all such variations and modifications. Thus, the methods and/or nanoparticles described herein are presently representative of the preferred embodiments, are intended to be exemplary and are not intended as limitations on the scope.

### REFERENCES

1 Neal, C. et al. Aging of Nanoscale Cerium Oxide in a Peroxide Environment: Its Influence on the Redox, Surface, and Dispersion Character. J. Phys. Chem. C 2021, 125, 27323-27334. https://doi.org/10. 1021/acs.jpcc.1c06279
2 Corsi, F.; Caputo, F.; Traversa, E.; Ghibelli, L. Not Only Redox: The Multifaceted Activity of Cerium Oxide Nanoparticles in Cancer Prevention and Therapy. Front. Oncol. 2018, 8, 309.
3 Dowding, J. M.; Song, W.; Bossy, K.; Karakoti, A.; Kumar, A.; Kim, A.; Bossy, B.; Seal, S.; Ellisman, M. H.; Perkins, G.; Self, W. T.; Bossy-Wetzel, E.; et al. Cerium Oxide Nanoparticles Protect against Aβ-Induced Mitochondrial Fragmentation and Neuronal Cell Death. Cell Death Differ. 2014, 21, 1622-1632.
4 Barbara, D. A.; Sandro, S.; Elisabetta, B.; Silvia Di, L.; Phani, R. A.; Stefano, F.; Fernanda, A.; Maria Paola, C.; Annamaria, C. Cerium Oxide Nanoparticles Trigger Neuronal Survival in a Human Alzheimer Disease Model by Modulating Bdnf Pathway. Curr. Nanosci. 2009, 5, 167-176.
5 Zgheib, C.; Hilton, S. A.; Dewberry, L. C.; Hodges, M. M.; Ghatak, S.; Xu, J.; Singh, S.; Roy, S.; Sen, C. K.; Seal, S.; Liechty, K. W. Use of Cerium Oxide Nanoparticles Conjugated with microRNA-146a to Correct the Diabetic Wound Healing Impairment. J. Am. Coll. Surg. 2019, 228, 107-115.
6 Chen, H. et al. Synthesis of Nanocrystalline Cerium Oxide Particles by the Precipitation Method. Ceramics International 2005, 31, 795-802. https://doi.org/10.1016/j.ceramint.2004.09.006.
7 Neal, C. et al. Metal-Mediated Nanoscale Cerium Oxide Inactivates Human Coronavirus and Rhinovirus by Surface Disruption. ACS Nano 2021, 15, 14544-14556. https://doi.org/10.1021/acsnano.1c04142
8 Heckert, E. G.; Karakoti, A. S.; Seal, S.; Self, W. T. The Role of Cerium Redox State in the Sod Mimetic Activity of Nanoceria. Biomaterials 2008, 29, 2705-2709.
9 Celardo, I. et al. Pharmacological Potential of Cerium Oxide Nanoparticles. Nanoscale 2011, 3, 1411-1420. DOI:10.1039/c0nr00875c
10 Lord, M. S. et al. Redox Active Cerium Oxide Nanoparticles: Current Status and Burning Issues. Small 2021, 17, 2102342. DOI:10.1002/smll.202102342
11 Hirst, S. M. et al. Anti-Inflammatory Properties of Cerium Oxide Nanoparticles. Small 2009 5(24): 2848-2856. DOI:10.1002/smll.200901048

What is claimed is:

1. A method for producing metal oxide nanoparticles comprising:
forming a solution by adding an oxidizing agent to an aqueous metal oxide; and
heating the solution formed for a preselected time at a preselected temperature in a preselected aeration environment.

2. The method according to claim 1, wherein heating the solution is carried out immediately after forming the solution.

3. The method according to claim 1, wherein heating the solution is carried out at about 24 hours after forming the solution.

4. The method according to claim 1, wherein the oxidizing agent is at least one of hydrogen peroxide, potassium dichromate, sodium hypochlorite, and calcium hypochlorite.

5. The method according to claim 1, wherein the aqueous metal oxide is at least one of a lanthanide series metal oxide, an actinide series metal oxide, and a titanium metal oxide.

6. The method according to claim 5, wherein the lanthanide series metal oxide is cerium.

7. The method according to claim 6, wherein the cerium is modified with a metal to form a hybrid composition.

8. The method according to claim 7, wherein the metal is at least one of a transition metal and a post-transition metal.

9. The method according to claim 8, wherein the transition metal is silver (Ag).

10. The method according to claim 1, wherein the preselected time is between about 24 hours and about 96 hours, the preselected temperature is between about 40° C. and about 90° C., and the preselected aeration environment ranges from about 5% oxygen to 100% oxygen.

11. The method according to claim 1, wherein the preselected time is 24 hours, and the preselected temperature is 70° C.

12. The method according to claim 1, wherein the preselected time is 96 hours, and the preselected temperature is 70° C.

13. The method according to claim 1, wherein the preselected time is 24 hours, and the preselected temperature is 90° C.

14. The method according to claim 1, further comprising titrating the solution to an acidic value prior to heating the solution.

15. Metal oxide nanoparticles produced according to the method of claim 1.

16. A method for producing cerium oxide nanoparticles (CNPs) comprising:
forming a solution by adding hydrogen peroxide to an aqueous cerium III salt; and
heating the solution formed for a preselected time at a preselected temperature in a preselected aeration environment.

17. The method according to claim 16, wherein the preselected time is between about 24 hours and about 96 hours, the preselected temperature is between about 40° C. and about 90° C., and the preselected aeration environment ranges from about 5% oxygen to 100% oxygen.

18. The method according to claim 16, further comprising titrating the solution to an acidic value prior to heating the solution.

19. The method according to claim 18, wherein the acidic value is a pH value of between about 2.0 and about 6.5.

20. The method according to claim 16, wherein the aqueous cerium III salt is modified with a metal to form a hybrid composition.

21. The method according to claim 20, wherein the metal is at least one of a transition metal and a post-transition metal.

22. The method according to claim 21, wherein the transition metal is silver (Ag).

23. Cerium oxide nanoparticles produced according to the method of claim 16.

24. The cerium oxide nanoparticles according to claim 23 modified with silver (Ag).

25. A composition comprising the cerium oxide nanoparticles (CNPs) produced according to the method of claim 16, wherein an amount of the composition ranges from approximately 1.5 ounces to approximately one gallon.

26. The method according to claim 1, wherein the aqueous metal oxide is a reducible rare earth metal.

27. The method according to claim 1, wherein the aqueous metal oxide is a lanthanide series metal oxide.

28. The method according to claim 8, wherein the metal is a transition metal.

29. The method according to claim 10, wherein the preselected temperature is between about 70° C. and about 90° C.

30. The method according to claim 14, wherein the acidic value is a pH value of between about 2.0 and about 6.5.

31. The method according to claim 1, further comprising titrating the solution to a pH value of about 2.5 prior to heating the solution.

32. A composition comprising the metal oxide nanoparticles produced according to the method of claim 1, wherein an amount of the composition ranges from approximately 1.5 ounces to approximately one gallon.

33. A composition comprising the metal oxide nanoparticles produced according to the method of claim 1, wherein an amount of the composition is at least one gallon.

34. A method for treating a subject having a condition responsive to metal oxide nanoparticles, the method comprising:
providing the metal oxide nanoparticles produced according to claim 1; and
administering the metal oxide nanoparticles to the subject, thereby treating the condition responsive to metal oxide nanoparticles in the subject.

35. The method according to claim 34, wherein the condition responsive to metal oxide nanoparticles is at least one of a malignant tumor, a benign tumor, a neurogenerative disease, a wound, and a condition causing hypoxia.

36. The method according to claim 35, wherein the wound is associated with diabetes.

37. The method according to claim 35, wherein the condition causing hypoxia is associated with ischemia.

38. The method according to claim 37, wherein the ischemia is associated with at least one of a transient ischemic attack (TIA) or a stroke.

39. A method for conferring a cytoprotective effect on cells, the method comprising:
providing the metal oxide nanoparticles produced according to claim 1; and
administering the metal oxide nanoparticles to the cells, thereby conferring the cytoprotective effect on the cells.

40. A kit for producing the metal oxide nanoparticles according to claim 1, wherein the kit comprises an oxidizing agent and an aqueous metal oxide.

41. The kit according to claim 40, wherein the kit further comprises a metal.

42. The kit according to claim 41, wherein the metal is silver (Ag).

43. The method according to claim 16, wherein the preselected temperature is between about 70° C. and about 90° C.

44. The method according to claim 16, wherein the preselected time is 24 hours and the preselected temperature is 70° C.

45. The method according to claim 16, wherein the preselected time is 96 hours and the preselected temperature is 70° C.

46. The method according to claim 16, wherein the preselected time is 24 hours and the preselected temperature is 90° C.

47. The method according to claim 16, wherein heating the solution is carried out immediately after forming the solution.

48. The method according to claim 16, wherein heating the solution is carried out at about 24 hours after forming the solution.

49. The method according to claim 16, further comprising titrating the solution to a pH value of about 2.5 prior to heating the solution.

50. The method according to claim 20, wherein the metal is a transition metal.

51. A composition comprising the ceriun oxide nanoparticles (CNPs) produced according to the method of claim 16, wherein an amount of the composition is at least one gallon.

52. A method for treating a subject having a condition responsive to cerium oxide nanoparticles (CNPs), the method comprising:
providing the cerium oxide nanoparticles (CNPs) produced according to claim 16; and
administering the cerium oxide nanoparticles (CNPs) to the subject, thereby treating the condition responsive to cerium oxide nanoparticles (CNPs) in the subject.

53. The method according to claim 52, wherein the condition responsive to metal oxide nanoparticles is at least one of a malignant tumor, a benign tumor, a neurogenerative disease, a wound, and a condition causing hypoxia.

54. The method according to claim 53, wherein the wound is associated with diabetes.

55. The method according to claim 53, wherein the condition causing hypoxia is associated with ischemia.

56. The method according to claim 55, wherein the ischemia is associated with at least one of a transient ischemic attack (TIA) or a stroke.

57. A method for conferring a cytoprotective effect on cells, the method comprising:
providing the cerium oxide nanoparticles (CNPs) produced according to claim 16; and
administering the cerium oxide nanoparticles (CNPs) to the cells, thereby conferring the cytoprotective effect on the cells.

58. A kit for producing the cerium oxide nanoparticles (CNPs) according to claim 16, wherein the kit comprises an oxidizing agent and an aqueous metal oxide.

59. The kit according to claim 58, wherein the kit further comprises a metal.

60. The kit according to claim 59, wherein the metal is silver (Ag).
